(19) United States
(12) Patent Application Publication
(10) Pub. No.: US 2026/0306711 A1
(43) Pub. Date: Oct. 8, 2026

(54) SUPERHYDROPHOBIC ANTIFOULING COMPOSITIONS AND APPLICATIONS THEREOF

(71) Applicant: University of Georgia Research Foundation, Inc., Athens, GA (US)

(72) Inventors: Hitesh Handa, Athens, GA (US); Elizabeth Brisbois, Athens, GA (US); Ekrem Ozkan, Ithaca, NY (US); Annalise Danielle-Marie Tucker, Athens, GA (US)

(21) Appl. No.: 19/639,392

(22) Filed: Apr. 6, 2026

Related U.S. Application Data
(60) Provisional application No. 63/785,040, filed on Apr. 8, 2025.

(51) Int. Cl. A61L 27/30; A61L 15/18; A61L 15/26; A61L 27/34; C08K 3/105; C08K 3/22; C09D 7/40; C09D 7/61; C09D 183/04

(52) U.S. Cl. CPC A61L 27/306; A61L 15/18; A61L 15/26; A61L 27/34; C08K 3/105; C08K 3/22; C09D 7/61; C09D 7/67; C09D 7/68; C09D 183/04; C08K 2003/2296; C08K 2201/005; C08K 2201/011

(57) ABSTRACT

Described herein are coating compositions having unique mechanical and physical properties. The coating compositions are composed of metal oxide nanoparticles, metal microparticles, metal particles selected from the group consisting of silver, copper, strontium, selenium, zinc, iron, nickel, and any combination thereof, and a silane compound. The compositions described herein are superhydrophobic, which keeps surfaces dry and clean. The compositions also produce nitric oxide (NO) when introduced to a subject. This dual functionality of the compositions enhances the material's antimicrobial performance, making it ideal for applications requiring both exceptional antibacterial efficacy and durability. The compositions can be applied to or incorporated in any article where it is desirable to reduce or prevent the development of biofilms and infection.

### CROSS-REFERENCE TO RELATED APPLICATIONS

This application claims the benefit of and priority to co-pending U.S. Provisional Patent Application No. 63/785,040, filed on Apr. 8, 2025, the contents of which are incorporated by reference herein in their entireties.

### STATEMENT REGARDING FEDERALLY SPONSORED RESEARCH OR DEVELOPMENT

This invention was made with government support under R01 HL157587 awarded by the National Institutes of Health. The government has certain rights in the invention.

### BACKGROUND

In the realm of human health, the initiation of infection can be triggered by the singular attachment of a bacterium, a seemingly minute event that catalyzes a complex cascade of physiological processes. When bacteria adhere to surfaces, they form biofilms that contribute to chronic infections and complications with medical devices¹⁻³. Each year, hospital-acquired infections account for 1.7 million cases and 99,000 deaths, with significant instances in urinary tract, surgical site, and bloodstream infections⁴. The overuse of antibiotics is escalating the rise of multi-drug-resistant bacteria, underscoring the urgent need for novel approaches to infection control⁵. At the forefront of biomedical research, innovative biomaterials and coatings are currently being engineered with remarkable antibacterial properties, revolutionizing the landscape of medical device fabrication and infection prevention. In an optimal scenario, antibacterial surfaces should possess both antibiofouling and bactericidal characteristics. Antibiofouling properties prevent bacteria from adhering and proliferating, while bactericidal properties actively reduce bacterial activity, employing chemical or physical mechanisms to eradicate bacteria⁶.

Antifouling surface modifications, such as superhydrophobicity, are gaining notable traction, emerging as a pivotal focus area across multiple industries, owing to their potential to mitigate biofouling and enhance material performance in diverse applications. Despite their effectiveness in repelling foulants and reducing adhesion, superhydrophobic surfaces are often limited in their ability to address planktonic microbial fouling.

### SUMMARY

Described herein are coating compositions having unique mechanical and physical properties. The compositions are composed of metal oxide nanoparticles, metal microparticles, metal particles selected from the group consisting of silver, copper, strontium, selenium, zinc, iron, nickel, and any combination thereof, and a silane compound. The compositions described herein are superhydrophobic, which keeps surfaces dry and clean. The compositions also produce nitric oxide (NO) when introduced to a subject. This dual functionality of the compositions enhances the material's antimicrobial performance, making it ideal for applications requiring both exceptional antibacterial efficacy and durability. The compositions can be applied to or incorporated in any article where it is desirable to reduce or prevent the development of biofilms and infection.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C show the fabrication and characterization of superhydrophobic samples. (A) Schematic illustration of the fabrication process for ZnO/10Cu, (B) FTIR for untreated ZnO nanoparticles, ODTMS, and ZnO superhydrophobic particles, (C) water contact angle and contact angle hysteresis measurements for superhydrophobic sample types. n 4; data presented as mean±SD.

FIGS. 2A-20 show the surface characterization of ZnO and Cu-Doped Samples. WCA (A, D, G, J, M), SEM (B, E, H, K, N), and AFM (C, F, I, L, O) images characterizing the surface of (A-C) control, (D-F) ZnO, (G-I) ZnO/1Cu, (J-L) ZnO/5Cu, and (M-O) ZnO/10Cu samples. SEM scale bars: 20 μm. AFM scale bars: 5 μm. n≥4; data presented as mean±SD.

FIGS. 3A-3M show durability testing of ZnO/10Cu Surface. (A) Sand drop test set-up, (B) 5 μL droplets of DI water on ZnO/10Cu post-sand drop, (C) water contact angle measurement of ZnO/10Cu post-sand drop, (D) ZnO/10Cu sample submerged in DI water with visible air plastron layer, (E) water-jet test on ZnO/10Cu, (F) tape peel test set-up, (G) 5 μL droplets of DI water on ZnO/10Cu after the first cycle of tape peeling, (H) water contact angle measurement of ZnO/10Cu after the first cycle of tape peeling, I) ZnO/10Cu performance to multiple cycles of tape-peeling, J) ZnO/10Cu after first cycle of blade scratching, (K) 5 μL droplets of DI water on ZnO/10Cu after the first cycle of blade scratching, (L) water contact angle measurement of ZnO/10Cu after the first cycle of blade scratching, (M) ZnO/10Cu performance to multiple cycles of blade scratching. n 4; data presented as mean±SD.

FIGS. 4A-4E provide nitric oxide generation measurements. (A) schematic and chemiluminescent method for detecting NO generation from materials, (B) schematic for a small selection of interactions between GSNO, GSH, and Cu ions¹⁹⁻²², (C) average peak NO flux, (D) accumulated NO normalized to surface area, (E) NO t₅₀ in response to material. *p<0.05 vs Control, tp<0.05 vs ZnO, *p<0.05 vs ZnO/1Cu, § p<0.05 vs ZnO/5Cu. n 6; data presented as mean±SD.

FIGS. 5A-5B NO-generating cell viability assays and relative cell viability analysis. (A) Schematic of NO-generating Cell Viability Assay, (B) Relative Cell viability for injected samples with labeled 70% ISO standard. n≥4; data presented as mean±SD.

FIGS. 6A-6B show the impact of NO-generating conditions on S. aureus bacterial adhesion and planktonic growth. (A) Adhered log S. aureus, (B) Planktonic log S. aureus. a (+) indicates the presence of physiological levels of GSNO and GSH, and a (−) indicates the absence. *p<0.05 vs Control (−), ^{†}p<0.05 vs Control (+), ^{‡}p<0.05 vs ZnO (−), ^{§}p<0.05 vs ZnO (+), ^{¶}p<0.05 vs ZnO/10Cu (−). n≥4; data presented as mean±SD.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” “having,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” includes, but is not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “alkyl group” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer.

The term “perfluoroalkyl group” refers to an alkyl group as defined herein where two or more hydrogen atoms on the alkyl group are substituted with a fluorine atom. In one aspect, all of the hydrogen atoms on the alkyl group are substituted with a fluorine atom. FIG. 11 provides an exemplary structure of a perfluoroalkyl group bonded to a siloxane group (—Si(OEt)₃).

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms or microbes (e.g., bacteria, fungi, virus, protozoans, algae, and the like). A substance having an antimicrobial characteristic can kill the microorganism and/or prevent or substantially prevent the growth or reproduction of the microorganism.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The terms “fungus” or “fungi” include, but are not limited to yeasts such as, for example, Candida albicans or other Candida spp. including C. glabrata, C. rugosa, C. parapsilosis, C. tropicalis, or C. dubliniensis Fungi can also include dermatophytes such as, for example, Trichophyton spp. and Microsporum spp. (e.g., T. rubrum, T. interdigitale, T. tonsurans, T. violaceum, T. concentricum, T. schoenleinii, T. soudanense, T. mentagrophytes, T. equinum, T. erinacei, T. verrucosum, M. audouinii, M. ferrugineum, M. canis, M. gypseum, M. nanum, and/or M. cookie).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, amelioration or palliation of the disease state, and remission (partial or total) whether detectable or undetectable.

As used herein, the term “prevent” or “preventing” refers to precluding, averting, obviating, forestalling, stopping, or hindering something from happening, especially by advance action.

As used herein, the term “reduce” or “reducing” refers to deceasing the degree or amount of an event. For example, reducing biofilm formation can mean lower the amount of biofilm formation when using the compositions described herein when compared to the amount of biofilm formation when the compositions described herein are not used.

As used herein, the term “nanoparticles” refers to particles having a diameter of less than 500 nm.

As used herein, the term “microparticles” refers to particles having a diameter of greater than or equal to 0.5 μm than 5 μm.

### Compositions and Applications Thereof

Described herein are compositions having unique mechanical and physical properties. The coating compositions are composed of metal oxide nanoparticles, metal microparticles, metal particles selected from the group consisting of silver, copper, strontium, selenium, zinc, iron, nickel, and any combination thereof, and a silane compound. Methods for preparing and using the compositions described herein are provided below.

A variety of metal oxides can be used to produce the compositions described herein. In one aspect, the metal oxide can be a transition metal oxide. In one aspect, the metal oxide nanoparticles and microparticles can be a mixed metal oxide (e.g., copper zinc iron oxide, Nickel zinc iron oxide, cerium(IV)-zirconium(IV) oxide, indium tin oxide, cobalt aluminum oxide). In one aspect, the metal oxide nanoparticles and microparticles can be zinc oxide, silicon oxide, aluminum oxide, titanium oxide, iron oxide, or any combination thereof. In another aspect, the metal oxide nanoparticles and microparticles are the same metal oxide. In another aspect, the metal oxide nanoparticles and microparticles are zinc oxide.

In one aspect, the metal oxide nanoparticles can have an average particle size of about 5 nm to about 100 nm, or about 5, nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, or 100 nm, where any value can be a lower and upper endpoint of a range (e.g., 10 nm to 30 nm).

In one aspect, the metal oxide microparticles can have an average particle size of about 0.5 μm to about 5 μm, or about 0.5 μm, 1.0 μm, 1.5 μm, 2.0 μm, 2.5 μm, 3.0 μm, 3.5 μm, 4.0 μm, 4.5 μm, or 5.0 μm, where any value can be a lower and upper endpoint of a range (e.g., 1.5 μm to 3.0 μm, etc.).

In one aspect, metal particles selected from the group consisting of silver, copper, strontium, selenium, zinc, iron, nickel, and any combination thereof. In one aspect, the metal particles are nanoparticles. In one aspect, the nanoparticles have an average particle diameter of about 10 nm to about 900 nm. In another aspect, the metal particles are silver nanoparticles having an average particle diameter of about 20 nm to about 800 nm, or about 20 nm, 50 nm, 100 nm, 150 nm, 200 nm, 250 nm, 300 nm, 350 nm, 400 nm, 450 nm, 500 nm, 550 nm, 600 nm, 650 nm, 700 nm, 750 nm, or 800 nm, where any value can be a lower and upper endpoint of a range (e.g., 50 nm to 150 nm). In another aspect, the metal particles are copper nanoparticles having an average particle diameter of about 5 nm to about 500 nm, or about 5 nm, 10 nm, 50 nm, 100 nm, 150 nm, 200 nm, 250 nm, 300 nm, 350 nm, 400 nm, 450 nm, or 500 nm, where any value can be a lower and upper endpoint of a range (e.g., 40 nm to 60 nm, etc.).

In one aspect, the weight ratio of metal oxide microparticles to metal oxide nanoparticles is from 5:1 to 1:5. In another aspect, the weight ratio of metal oxide microparticles to metal oxide nanoparticles is 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, or 1:5, where any value can be a lower and upper endpoint of a range (e.g., 1.5:1 nm to 1:1.5 nm, etc.).

In one aspect, the weight ratio of the sum of the metal oxide microparticles and metal oxide nanoparticles to the metal particles is from 8:1 to 99:1. In another aspect, the weight ratio of the sum of the metal oxide microparticles and metal oxide nanoparticles to the metal particles is 8:1, 8.5:1, 9:1. 9.5:1, 10:1, 20:1, 30:1, 40:1, 50:1, 60:1, 70:1, 80:1, 90:1, 95:1, or 99:1, where any value can be a lower and upper endpoint of a range (e.g., 8.5:1 nm to 9.5:1 nm, etc.).

The compositions described herein include a silane compound. In one aspect, the silane compound is an alkyl trialkoxy silane. In another aspect, the silane compound has the formula SiR¹(OR²)₃, wherein R¹ is a C10 to C20 alkyl group and each R² is a C1 to C10 alkyl group. In another aspect, the silane compound is octadecyl(trimethoxy)silane. In one aspect, the weight ratio of the sum of the metal oxide microparticles and metal oxide nanoparticles to the silane compound is from 20:1 to 100:1. In another aspect, the weight ratio of the sum of the metal oxide microparticles and metal oxide nanoparticles to the silane compound is 20:1, 25:1, 30:1. 35:1, 40:1, 45:1, 50:1, 55:1, 60:1, 65:1, 70:1, 75:1, 80:1, 85:1, 90:1, or 100:1, where any value can be a lower and upper endpoint of a range (e.g., 55:1 nm to 65:1 nm, etc.). In one aspect, the weight ratio of the sum of the metal particles to the silane compound is from 1:1 to 10:1. In another aspect, the weight ratio of the metal particles to the silane compound is 1:1, 2:1, 3:1, 4:1, 5:1, 60:1, 7:1, 8:1, 9:1, or 10:1, where any value can be a lower and upper endpoint of a range (e.g., 5:1 nm to 7:1 nm, etc.).

In certain aspects, the compositions described herein include one or more polymers. In one aspect, the polymer is a thermoplastic silicone-polycarbonate-urethane, a polyester, a polyamide, a polyalkylene glycol (e.g., polyethylene glycol, polypropylene glycol, or a polyethylene/polypropylene glycol copolymer), polyvinyl alcohol, polyurethane, sodium alginate, chitosan, cellulose, collagen, chondroitin, gelatin, silk fibroin, eggshell membrane, albumin, wheat bran, arabinoxylan, or any combination thereof.

In one aspect, the polymer is a polysiloxane. In one aspect, the polysiloxane is a linear or branched polysiloxane. In another aspect, the first polymer and the second polymer are the same polysiloxane.

In one aspect, the polysiloxane has the structure I

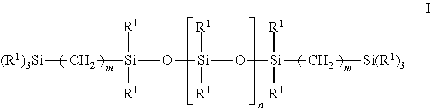

wherein each R¹ is independently selected from alkyl, aryl, alkenyl, hydrogen, alkoxy;
m is an integer from 1 to 10; and
n is from about 2,500 to about 27,000.

In one aspect, each R¹ in structure I is a methyl group. In another aspect, each m in structure I is 3. In another aspect, n in structure I is 2,500, 5,000, 7,500, 10,000, 12,500, 15,000, 17,500, 20,000, 22,500, 25,00, or 27,000, where any value can be a lower and upper endpoint of a range (e.g., 5,000 to 17,500).

In one aspect, the polysiloxane is a room temperature vulcanization (RTV) material. In one aspect, the polysiloxane comprises a polydimethylsiloxane, a polydiethylsiloxane, a polydipropylsiloxane, or a polydiphenylsiloxane. In another aspect, the polymer is polydimethylsiloxane. In one aspect, the polysiloxane is polydimethylsiloxane sold under the tradename Sylgard™ 184, which is a silicone elastomer manufactured by Dow.

In one aspect, the compositions described herein can include an organic solvent can include an alcohol. In one aspect, the alcohol can be a C₁ to C₁₀ alcohol. In another aspect, the organic solvent can include, but is not limited to, methanol, ethanol, propanol, isopropanol, butanol, or any combination thereof. In another aspect, the organic solvent can be a hydrocarbon such as, for example, hexane.

In one aspect, the compositions described herein do not include a fluorosilane, which pose health and environmental risks. For example, the compounds described herein do not include a compound having a perfluoroalkyl group covalently bonded to a siloxane group. In one aspect, the perfluorolkylsiloxane can have the formula R²—Si(OR¹)₃, wherein R¹ is a substituted or unsubstituted C₁-C₂₀ alkyl group, and R² is a C₁-C₂₀ perfluoroalkyl group.

In another aspects, depending upon the application of the compositions the material can include one or more bioactive agents. In one aspect, the bioactive material includes, but is not limited to, an antibiotic, a pain reliever, an immune modulator, a growth factor, an enzyme inhibitor, a hormone, a messenger molecule, a cell signaling molecule, a receptor agonist, an oncolytic virus, a chemotherapy agent, an anti-angiogenic agent, a receptor antagonist, a nucleic acid, or any combination thereof.

The coating compositions described herein can be made by admixing metal oxide nanoparticles, metal oxide microparticles, metal particles selected from the group consisting of silver, copper, strontium, selenium, zinc, iron, nickel, and any combination thereof, and a silane compound in a solvent. In one aspect, the metal oxide nanoparticles, metal oxide microparticles, metal particles, and silane compound each in selected amounts are mixed in a solvent (e.g., a hydrocarbon solvent with or without water). In certain aspects, the polymer (e.g., polydimethylsiloxane) is added to the mixture. Additional agents such as, for example, a curing agent, can be added to the mixture. The Examples provide non-limiting procedures for making the compositions described herein.

The coating compositions described herein are very hydrophobic (i.e., superhydrophobic). Superhydrophobicity refers to the remarkable property of certain surfaces to repel water to such an extent that water droplets bead up and roll off rather than spreading out. The degree of hydrophobicity can be measured by the contact angle of the coating. In another aspect, the coating and coated article can have a contact angle hysteresis of from about 0.1 degrees to about 10 degrees, or about 0.1 degrees, 0.5 degrees, 1.0 degrees, 1.5 degrees, 2.0 degrees, 2.5 degrees, 3.0 degrees, 3.5 degrees, 4.0 degrees, 4.5 degrees, 5.0 degrees, 5.5 degrees, 6.0 degrees, 6.5 degrees, 7.0 degrees, 7.5 degrees, 8.0 degrees, 8.5 degrees, 9.0 degrees, 9.5 degrees, or 10.0 degrees, where any value can be a lower and upper endpoint of a range (e.g., 1.5 degrees to 4.0 degrees, etc.). In another aspect, the composition has a static water contact angle of greater than 150 degrees. In another aspect, the composition has a static water contact angle of 150 degrees, 160 degrees, 170 degrees, 180 degrees, 190 degrees, or 200 degrees, where any value can be a lower and upper endpoint of a range (e.g., 160 degrees to 180 degrees, etc.). With the high degree of hydrophobicity, the coatings and articles composed of the compositions described herein can repel various liquids including, but not limited to, water, milk, coffee, juice, and blood.

In another aspect, the compositions described herein possess high surface roughness, which also contributes to increased superhydrophobicity. In one aspect, the composition has a root mean square roughness (Rq) of at least 225 nm. In another aspect, composition has a root mean square roughness (Rq) of 225 nm, 225 nm, 225 nm, 225 nm, 225 nm, 225 nm, 225 nm, or 225 nm, where any value can be a lower and upper endpoint of a range (e.g., 300 nm to 350 nm, etc.).

The compositions described herein when incorporated into an article are capable of maintaining their unique physical and chemical properties (e.g., superhydrophobicity) when exposed to mechanical agitation. For example, the coatings are robust when subjected to different types of harsh mechanical agitation, damage, or scratching (e.g., finger-wiping, knife-scratching, tape-peeling, hand-kneading, hand-rubbing, bending, etc.).

The compositions described herein can be applied to and/or incorporated into a variety of articles where it is desirable to repel liquids and prevent biofouling. In one aspect, the article is produced by applying the composition as described herein to at least one surface of the article and, when applicable removing any solvent from the composition to produce a coated article. The composition can be applied to the article using techniques known in the art such as, for example, dipping or spraying. In one aspect, a single coating can be applied to the article. In other aspects, multiple coatings can be sequentially applied to the article. After the coating composition is applied to the article, any solvent that may be present is removed. In one aspect, the organic solvent can be removed by evaporation. In one aspect, the organic solvent can be removed by heating the coated article at a temperature of from about 80° C. to about 120° C. In one aspect, heating is sufficient to remove at least 80%, at least 85%, at least 90%, at least 95%, at least 99%, or 100% of the solvent.

In one aspect, the article comprises a polymeric grade material, a surface or article in a hospital or medical facility, a metal or a surface of a metal, a glass or a surface of a glass, or a surface in an automobile, boat, a boat surface, or aircraft. In another aspect, the article to be coated is an implantable device including, but not limited to, a catheter, a cannula, tubing, a suture, an artificial heart valve, a graft, a stent, circuitry, or a joint prostheses. In another aspect, article is a wound dressing including, but not limited to a bandage, gauze, pad.

In another aspect, one or more components of an article can be fabricated with the composition described herein. In one aspect, the composition can be poured into a mold. When a solvent is present in the composition, the solvent can be removed to produce a molded article composed of the composition described herein. In other aspect, the article can include a gel or foam, where the composition described herein is formulated with components used to produce gels or foams.

The compositions described herein can be applied to and/or incorporated into any article or surface where it is desirable to reduce or prevent biofouling (e.g. growth of bacteria, adhesion of platelets, adhesion of fibrinogen). Biofilm and thrombus formation on surfaces results in significant morbidity and mortality worldwide, which highlights the importance of the development of efficacious fouling-prevention approaches. Provided herein are highly robust and superhydrophobic coatings with outstanding multi-liquid repellency, bactericidal performance, and extremely low bacterial and blood adhesion.

In addition to possessing superhydrophobic properties, the compositions described herein when introduced into a subject can generate nitric oxide in vivo. Nitric oxide (NO) is a RONS produced endogenously from L-arginine by a class of enzymes known as nitric oxide synthases (NOS) and several cofactors. NO possesses unique antimicrobial abilities effective against bacterial species, as well as a broad spectrum of other pathogenic microorganisms, including viruses, fungi, and parasites. A diverse array of S-nitrosothiols (RSNOs) such as S-nitrosoglutathione (GSNO), S-nitrosoalbumin, and S-nitrosocysteine are present in the bloodstream. The compositions described can produce NO in vivo when the composition comes into contact with the RSNO. By combining superhydrophobicity with NO generation, the compositions described herein not only catalyze high levels of NO release from endogenous NO donors like RSNOs but also keep the surface dry and clean. This dual functionality enhances the material's antimicrobial performance, making it ideal for applications requiring both exceptional antibacterial efficacy and durability.

In one aspect, the compositions described herein are useful in applications where it is desirable to reduce or prevent biofouling. Implantable medical devices are a leading cause of infection such as nosocomial infections. Implantable devices coated with or constructed with the compositions described herein can reduce or prevent biofouling in a subject when the device is introduced into the subject. In one aspect, the compositions described herein can reduce or prevent bacterial growth on a surface of an implantable device. In another aspect, the compositions described herein can reduce or prevent biofilm formation on a surface of an implantable device. In another aspect, the compositions described herein can reduce or prevent fibrinogen formation on a surface of an implantable device.

In one, the implantable device is a urinary catheter, artificial heart valve, a vascular catheter, a graft, or a stent. In other aspects, the device is intended to contact human blood or tissue. In one aspect, the device is a hemodialysis device or a component thereof. The coating compositions described herein are biocompatible (e.g., with fibroblast cells), which makes them useful in implantable medical devices.

In another aspect, the coating compositions described herein are useful in applications where it is desirable to reduce or prevent biofouling on polymeric medical grade materials (e.g. silicone, polyvinyl chloride (PVC), polyurethane (PU)). In other aspects, the coating compositions described herein are useful in applications where it is desirable to reduce or prevent biofouling on metals (e.g. steel, titanium). In other aspects, the compositions described herein are useful in applications where it is desirable to reduce or prevent biofouling on hospital touch surfaces (e.g. bed rails, bed frames, and handles).

In one aspect, the coating compositions described herein can prevent the growth of bacteria on an article, in which the method includes applying the composition as above to at least one surface of the article. The coated article can prevent about at least 80%, at least 85%, at least 90%, at least 95%, at least 99%, or 100% of the growth of bacteria when compared to an uncoated article.

In one aspect, the coating compositions described herein can prevent the adhesion of fibrinogen on an article, in which the method includes applying the composition as above to at least one surface of the article. The coated article can prevent about at least 80%, at least 85%, at least 90%, at least 95%, at least 99%, or 100% of the adhesion of fibrinogen when compared to an uncoated article.

In one aspect, the coating compositions described herein can prevent the adhesion of platelets on an article, in which the method includes applying the composition as above to at least one surface of the article. The coated article can prevent about at least 80%, at least 85%, at least 90%, at least 95%, at least 99%, or 100% of the adhesion of platelets when compared to an uncoated article.

In one aspect, the compositions described herein are useful in promoting the healing of a number of different wounds. Proper wound management requires multi-faceted healing platforms that regulate inflammation, stimulate tissue growth, promote blood flow, and prevent infection. Microbial burden becomes a major issue in wound management, especially in delayed healing and the development of chronic wounds (e.g., diabetic foot, venous leg, and pressure ulcers).

Skin and soft tissue infections (SSTIs) result from microbial contamination of the skin, tissue, or muscles and is common to open wounds resulting from cuts, punctures, or burns. SSTIs can range from mild infections to complicated, severe infections depending on the patient's state and opportunistic microorganisms. Improper care of an open wound can lead to microbial invasion of community-acquired methicillin-resistant Staphylococcus aureus or streptococci, resulting in difficult-to-treat SSTIs. Involvement of such antibiotic-resistant Gram-positive strains increases the difficulty of treatment, and can significantly affect patient outcomes, leading to increased costs for chronic wound treatment and extended hospitalization. In this sense, initial wound care and management is critical to prevent the incidence of SSTIs.

Chronic wounds, common in the elderly population, do not progress through a normal process of repair, often remaining open for an extended period. Normally, the wound healing process is hindered by microorganisms and/or necrotic tissue by producing proinflammatory cytokines, elevating matrix metalloproteinases, and attracting excessive neutrophils. Thus, destroying or inactivating the building blocks necessary for normal wound healing. This also allows bacteria to proliferate and colonize the wound by constructing protected colonies or biofilms. More than 90% of chronic wounds contain bacteria existing within a biofilm construct. Biofilms prevent antibiotics and immune cells from penetrating the bacteria colony, giving added resistance power to bacteria to tolerate harsh conditions. Chronic wounds become more complicated with biofilm formation resulting in wounds that may take several years to heal. Therefore, early diagnosis can allow proper wound care treatment to avoid the incidence of chronic wounds.

SSTIs remain a broad clinical challenge to clinicians where antibiotic use is also heavily debated for its benefits/drawbacks to wound repair. Reducing bioburden is a key component to wound recovery through the normal phases of healing (i.e., hemostasis, inflammation, proliferation, and remodeling). However, avoiding chronic wound development is key in diverse patient groups (e.g., diabetes, poor circulation, malnutrition, etc.) wherein these technologies can have broad limitations (e.g., infiltration of inflammatory cells, lack of fibroblast migration, and low amounts of extracellular matrix components).

In one aspect, the compositions described herein are effective in reducing or preventing bacterial infection (e.g., Pseudomonas aeruginosa and Methicillin Resistant Staphylococcus aureus) when applied to the wound. In another aspect, the compositions described herein are effective in reducing or preventing biofilm formation, which can prevent antibiotics and immune cells from penetrating the bacteria colony in the wound.

In one aspect, the wound treatment materials described herein can reduce or prevent inflammation at or near the wound. Not wishing to be bound by theory, nitric oxide produced in vivo by the compositions described herein can promote M2 macrophage polarization, leading to healing, fixing, antiinflammation.

The compositions are effective in treating a number of significant wounds. In one aspect, the wound is the result of a traumatic injury. Traumatic injury is the leading cause of death for Americans up to the age of 45 years and the fourth leading cause of death overall. Uncontrolled trauma-related hemorrhage is the leading cause of death among critically injured patients. Infectious complications often result from extensive wound contamination in traumatic injury patients, and can lead to sepsis and organ failure.

In one aspect, the wound is the result of a surgical procedure or intervention. Surgical intervention often results in trauma to tissues and organs. Up to 3% of patients undergoing surgery will experience infection at or adjacent to the surgical site, extending hospitalization by more than a week on average.

In one aspect, the wound is a chronic wound. Chronic wound management is a broad challenge, with the mainstay of treatment being the TIME principle: tissue debridement, infection control, moisture balance, and edges of wound. More than 4.5 million Americans are affected by chronic lower extremity ulcers and related chronic wounds annually, with these ulcers typically lasting 12 to 13 months and having a high risk of recurrence. In one aspect, the chronic wound is a skin ulcer such as, for example, a diabetic ulcer, pressure ulcer, or venous ulcer. In another aspect, the wound is a burn.

Depending upon the nature of the wound, the compositions described herein can be used alone (e.g., a wound packing) or incorporated into other wound dressing articles such as, for example, a bandage, gauze, pad, a gel, or a foam.

### Aspects

Aspect 1. A composition comprising
(a) metal oxide nanoparticles;
(b) metal oxide microparticles;
(c) metal particles selected from the group consisting of silver, copper, strontium, selenium, zinc, iron, nickel, and any combination thereof; and
(d) a silane compound.

Aspect 2. The composition of Aspect 1, wherein the metal oxide nanoparticles comprise zinc oxide, silicon oxide, aluminum oxide, titanium oxide, iron oxide, or any combination thereof.

Aspect 3. The composition of Aspect or 1 or 2, wherein the metal oxide microparticles comprise zinc oxide, silicon oxide, aluminum oxide, titanium oxide, iron oxide, or any combination thereof.

Aspect 4. The composition of any one of Aspects 1-3, wherein the metal oxide nanoparticles have an average particle size of from about 5 nm to about 100 nm.

Aspect 5. The composition of any one of Aspects 1-3, wherein the metal oxide microparticles have an average particle size of from about 0.5 μm to about 5 μm.

Aspect 6. The composition of any one of Aspects 1-5, wherein the metal oxide nanoparticles and the metal oxide nanoparticles are each zinc oxide.

Aspect 7. The composition of any one of Aspects 1-3, wherein the metal particles are metal nanoparticles.

Aspect 8. The composition of Aspect 7, wherein the metal nanoparticles have an average particle size of from about 10 nm to about 100 nm.

Aspect 9. The composition of any one of Aspects 1-8, wherein the metal particles comprise copper nanoparticles having an average particle size of from about 5 nm to about 500 nm.

Aspect 10. The composition of any one of Aspects 1-9, wherein a weight ratio of metal oxide microparticles to metal oxide nanoparticles is from 5:1 to 1:5.

Aspect 11. The composition of any one of Aspects 1-10, wherein a weight ratio of the sum of the metal oxide microparticles and metal oxide nanoparticles to the metal particles is from 8:1 to 99:1.

Aspect 12. The composition of any one of Aspects 1-11, wherein composition further comprises a polymer, wherein the polymer comprises a thermoplastic silicone-polycarbonate-urethane, silicone-polyether-urethane, a polyester, a polyamide, a polyalkylene glycol, polyvinyl alcohol, polyurethane, sodium alginate, chitosan, cellulose, collagen, chondroitin, gelatin, silk fibroin, eggshell membrane, albumin, wheat bran, arabinoxylan, poly(lactic-co-glycolic acid), or any combination thereof.

Aspect 13. The composition of Aspect 12, wherein the polymer comprises a polysiloxane.

Aspect 14. The composition of Aspect 13, wherein the polysiloxane comprises a linear or branched polysiloxane.

Aspect 15. The composition of Aspect 13 or 14, wherein the polysiloxane comprises a polydimethylsiloxane, a polydiethylsiloxane, a polydipropylsiloxane, or a polydiphenylsiloxane.

Aspect 16. The composition of any one of Aspects 1-15, wherein the silane compound is an alkyl trialkoxy silane.

Aspect 17. The composition of any one of Aspects 1-15, wherein the silane compound has the formula SiR¹(OR²)₃, wherein R¹ is a C10 to C20 alkyl group and each R² is a C1 to C10 alkyl group.

Aspect 18. The composition of any one of Aspects 1-13, wherein the silane compound is octadecyl(trimethoxy)silane.

Aspect 19. The composition of any one of Aspects 1-18, wherein the composition further comprises an organic solvent.

Aspect 20. The composition of Aspect 19, wherein the organic solvent comprises an alcohol or a hydrocarbon.

Aspect 21. The composition of Aspect 19, wherein the organic solvent comprises a C₁ to C₁₀ hydrocarbon.

Aspect 22. A composition produced by mixing metal oxide nanoparticles, metal oxide microparticles, metal particles selected from the group consisting of silver, copper, strontium, selenium, zinc, iron, nickel, and any combination thereof, and a silane compound in a solvent.

Aspect 23. The composition of any one of Aspects 1-22, wherein the composition does not include a perfluorolkylsiloxane.

Aspect 24. The composition of any one of Aspects 1 to 23, wherein the composition has a contact angle hysteresis of less than 10 degrees.

Aspect 25. The composition of any one of Aspects 1 to 24, wherein the composition has a static water contact angle of greater than 150 degrees.

Aspect 26. The composition of any one of Aspects 1 to 25, wherein the composition has a root mean square roughness (Rq) of at least 225 nm.

Aspect 27. The composition of any one of Aspects 1 to 26, wherein the composition is biocompatible.

Aspect 28. An article comprising the composition of any one of Aspects 1 to 27.

Aspect 29. The article of Aspect 28, wherein the article comprises at least one surface comprising a coating of the composition.

Aspect 30. The article of Aspect 28, wherein one or more components of the article are fabricated with the composition.

Aspect 31. The article of any one of Aspects 28 to 30, wherein the article comprises a polymeric grade material, a surface or article in a hospital or medical facility, or a surface in an automobile, boat, a metal or a surface of a metal, a glass or surface of a glass, or aircraft.

Aspect 32. The article of any one of Aspects 26 to 28, wherein the article comprises a medical device.

Aspect 33. The article of Aspect 32, wherein the article is an implantable device.

Aspect 34. The article of Aspect 32, wherein the article is a catheter, a cannula, tubing, a suture, an artificial heart valve, a graft, a stent, circuitry, or a joint prostheses.

Aspect 35. The article of any one of Aspects 28 to 30, wherein the article comprises a dressing.

Aspect 36. The article of Aspect 35, wherein the dressing comprises a bandage, gauze, pad, a gel, or a foam.

Aspect 37. A method for preventing the growth of bacteria on an article when the article is introduced in a subject, the method comprising introducing the article of any one of Aspects 28 of 36 to the subject.

Aspect 38. A method for preventing the adhesion of fibrinogen on an article when the article is introduced in a subject, the method comprising introducing the article of any one of Aspects 28 of 36 to the subject.

Aspect 39. A method for preventing the adhesion of platelets on an article when the article is introduced in a subject, the method comprising introducing the article of any one of Aspects 28 of 36 to the subject.

Aspect 40. A method for preventing the adhesion of platelets on an article when the article is introduced in a subject, the method comprising introducing the article of any one of Aspects 28 of 36 to the subject.

Aspect 41. A method for producing nitrogen oxide (NO) in a subject, the method comprising introducing the article of any one of Aspects 28 of 36 to the subject.

Aspect 42. A method for treating a wound, the method comprising applying the article of any one of Aspects 28 of 36 to the wound.

Aspect 43. The method of Aspect 42, wherein the wound treatment material reduces or prevents inflammation at or near the wound.

Aspect 44. The method of Aspect 42 or 43, wherein the wound is the result of a traumatic injury or surgical intervention.

Aspect 45. The method of Aspect 42 or 43, wherein the wound is a chronic wound.

Aspect 46. The method of Aspect 43, wherein the chronic wound is a diabetic ulcer, pressure ulcer, or venous ulcer.

Aspect 47. The method of Aspect 42, wherein the wound is a burn to the skin.

Aspect 48. A method for preventing the growth of bacteria on an article, the method comprising applying the coating composition of any one of Aspects 1 to 27 to at least one surface of the article.

Aspect 49. A method for preventing the adhesion of fibrinogen on an article, the method comprising applying the coating composition of any one of Aspects 1 to 27 to at least one surface of the article.

Aspect 50. A method for preventing the adhesion of platelets on an article, the method comprising applying the coating composition of any one of Aspects 1 to 27 to at least one surface of the article.

### Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

Zinc oxide (ZnO) nanoparticles (10-30 nm) and copper (Cu) nanoparticles (40-60 nm) were sourced from Skyspring Nanomaterials Inc. (Houston, USA), while ZnO microparticles (<5 μm) and other materials such as octadecyl(trimethoxy)silane (ODTMS), n-hexane, a mixture of isomers, sodium nitrite (NaNO₂), hydrochloric acid (HCl), dimethyl sulfoxide (DMSO), sodium chloride, potassium chloride, sodium phosphate monobasic, sodium phosphate dibasic, Copper Assay kit (MAK127), and Luria-Bertani (LB) broth and agar were acquired from Sigma Aldrich (St. Louis, MO). Zinc assay kit (ab176725) was obtained from abcam (Cambridge, MA). Sylgard-184 polydimethylsiloxane base and curing agent (PDMS) were obtained from Ellsworth Adhesives (USA). Reduced L-glutathione was purchased from GoldBio, Inc (St. Louis, MO). Staphylococcus aureus (S. aureus) (ATCC 6538) and 3T3 mouse fibroblast cells (ATCC CRL-1658) were procured from American Tissue Culture Collection (ATCC, Manassas, VA, USA). Dulbecco's Modified Eagle Medium (DMEM), penicillin-streptomycin (PS), and Ethylenediaminetetraacetic acid (EDTA) were sourced from Fischer Scientific. Cell culture-grade calcium and magnesium-free phosphate buffer saline (CMF-PBS, 1×) and 0.25% Trypsin were sourced from Corning Incorporated (Corning, NY). Fetal bovine serum (FBS) was sourced from VWR International (Radnor, PA). 3-(4,5-dimethylthiazol-2-yl)-2,5-diphenyltetrazolium bromide (MTT) reagent sourced from Roche (Basel, CH). DI water was utilized in all appropriate experiments; all chemicals were analytical-grade reagents and utilized without further purification. Phosphate-buffered saline (PBS) was prepared with 139 mM sodium chloride, 2.68 mM potassium chloride, 1.8 mM sodium phosphate monobasic, and 8.2 mM sodium phosphate dibasic. The pH of PBS was verified to be 7.4 at 25° C. using a calibrated pH meter. Calibration was performed with standard buffer solutions of known pH prior to measurement.

### Fabrication of Superhydrophobic Particles and Coating Solutions

A coating precursor solution was created using specified amounts of ZnO, Cu, ODTMS, n-hexane, and DI water, as tabulated in Table 1. In short, ZnO microparticles, ZnO and Cu nanoparticles, ODTMS, and DI water were added to 20 g of n-hexane and allowed to mix at 300 rpm for 8 h.

TABLE 1
Ratios of Components in Coating Precursor Solution.

| | n- | | ZnO | ZnO | Cu | | |
|---|---|---|---|---|---|---|---|
| | hexane | ODTMS | μp | np | np | DI | ZnO:Cu |
| Formulation | (g) | (g) | (g) | (g) | (g) | (μL) | Ratio |
| ZnO | 20 | 0.2 | 6 | 6 | — | 10 | 100:0 |
| ZnO/1Cu | 20 | 0.2 | 6 | 6 | 0.12 | 10 | 99:1 |
| ZnO/5Cu | 20 | 0.2 | 6 | 6 | 0.60 | 10 | 95:5 |
| ZnO/10Cu | 20 | 0.2 | 6 | 6 | 1.2 | 10 | 90:10 |

*Prior to addition of PDMS in final fabrication step of coating solutions

To create the final coating solution, 10 wt % Sylgard-184 silicone base and curing agent (10:1 ratio of base to curing agent) were added to approximately 20 g of precursor solution. This final coating solution was mixed at 200 rpm for 30 min, vortexed for 1 min, ultrasonicated for 5 min, and vortexed again for 1 min.

Neat Sylgard-184 PDMS was cured and used as the base polymer for film coating. The base films (8 mm in diameter, ~0.3 mm thick) were dipped into the final coating solution, and any excess solution was gently shaken off. Films were then cured at 100° C. for 2 h to obtain superhydrophobic samples.

Before Sylgard-PDMS addition, the residual precursor solution was allowed to evaporate in a fume-hood overnight, dried at 100° C. for 1 h, and collected to be used in characterization studies as described. Sylgard-184 PDMS base films were weighed before and after dip-coating and curing to determine the mass of the cured coating on the surface, which averaged 6.46±0.98 mg per sample.

Before optimizing final samples with varying weights of Cu, an investigation was conducted on the amount of base metal-oxide components. Superhydrophobic ZnO samples were created with varying amounts of nanoparticles to microparticles to identify concentrations that maintained superhydrophobicity, but minimal surface coating cracking. Representative images are depicted. See Table 2 for the ratios explored.

TABLE 2
Nanoparticle and Microparticle Ratio for Coating Optimization.

| | n-hexane | ODTMS | ZnO μp | ZnO np | DI |
|---|---|---|---|---|---|
| Formulation | (g) | (g) | (g) | (g) | (μL) |
| ZnO 0:100 | 20 | 0.2 | 0 | 12 | 10 |
| ZnO 25:75 | 20 | 0.2 | 3 | 9 | 10 |
| ZnO 75:25 | 20 | 0.2 | 9 | 3 | 10 |
| ZnO 100:0 | 20 | 0.2 | 12 | 0 | 10 |
| ZnO (50:50) | 20 | 0.2 | 6 | 6 | 10 |

### Surface Characterization

### Confirmation of Silanization Via Fourier Transform Infrared Spectroscopy (FTIR)

Fourier transform infrared spectroscopy (FTIR) using a Spectrum 3 spectrometer (Perkin-Elmer) was used to confirm silanization of nanoparticle and microparticle systems. A quantitative potassium bromide (KBr) method was used with the bare particles and the dried residual precursor solution while Universal Attenuated Total Reflectance (UATR) FTIR method was used to characterize neat silane. Samples were analyzed in a range of 4000 to 650 cm⁻¹ with a resolution of 4 cm⁻¹ and a total of 128 scans recorded per sample type. A background spectrum was used before each sample for all FTIR methods.

### Antiwetting Analysis: Water Contact Angle (WCA) and Contact Angle Hysteresis (CAH)

Antiwetting properties of control PDMS and superhydrophobic sample types were assessed using a contact angle goniometer (Ossila) and image processing software (SURFTENS). To measure the static water contact angle of the surface, 8 mm samples were placed upon a stage positioned in front of a camera before a 5 μL droplet of DI water was dispensed onto the surface of the sample. An image of the sample with the water droplet was taken and the water contact angle was processed using the aforementioned software.

Contact angle hysteresis (CAH) was calculated using the advancing and receding angles water droplets make with the surface of the samples using Equation 1. Using a Hamilton syringe, a 5 μL droplet was dispensed and withdrawn from the surface of the samples. Videos were recorded and subsequently processed, allowing for analysis of the relative angles. Final data for static water contact angle and hysteresis measurements are reported as the mean±standard deviation (SD) with n≥4 for each sample type.

Contact Angle Hysteresis (°)=Advancing Contact Angle (°)-Receding Contact Angle (°) (1)

### Surface Morphology Analysis Via Microscopy

Surface characterization was analyzed via scanning electron microscopy (SEM) using a FEI Teneo SEM (FEI Co.) to observe the surface texturization and homogeneity after the metallic dipcoats. Elemental mapping was also conducted on each sample to detect the ZnO and Cu particle dispersion on the surface of the materials using Energy Dispersive X-Ray spectroscopy (EDS, Oxford Instruments). EDS images were analyzed using an Aztec application set to detect Cu, Zn, Carbon, Oxygen, and Silicon on the surface. All images were taken with a magnification of 1000× or 7500× under a 10.00 kV voltage. Before any imaging, the samples were sputter coated with a 10 nm coating of gold-palladium to enhance readability on the SEM using a Leica sputter coating instrument. Cross-section images of final sample ZnO/10Cu were also taken and analyzed for thickness of final coating.

For Atomic Force Microscopy (AFM) analysis, control PDMS and the ZnO/10Cu surface were imaged for their topography and surface roughness measurements. AFM was performed using a Multimode8 Tapping microscope (Bruker) at room-temperature. Topography images were obtained by scanning samples in tapping mode with a scan size of 20 μm by 20 μm, respectively. Scan rates of 0.1 Hz and resolutions of 256 samples per line were used to generate representative images.

LTESPA-V2 cantilever tips (Bruker) with spring constant 42 N/m were used for all imaging. All images were flattened and plane-fitted using NanoScope Analysis software (Bruker); all films were 2^{nd} order flattened. Roughness measurements were calculated using n=4 of (3 μm×3 μm) images to represent four different locations per sample type.

### Mechanical Durability Testing

To evaluate superhydrophobic stability, both qualitative and quantitative tests were conducted. Qualitative analyses involved sand abrasion, tape, and scratch tests as described herein.

A sand-drop test was conducted on the material. In short, the surface was immobilized onto an inclined block, a funnel was placed directly above the surface, and sand (10 g) was dropped from a height of 20 cm. Any residual sand is brushed off the final surface, and the water contact angle after one cycle is measured.

The sample was submerged in DI water and placed under a water jet for qualitative observations of the surface's superhydrophobicity.

The tape-peel test was conducted by placing a piece of tape on top of the surface, adding a 500 g weight for ~15 s, removing the weight, and peeling the tape from the surface. After the tape was peeled off, a static gun was administered to the surface prior to water contact angle and hysteresis measurements. A razor blade was used to scratch the surface of the material repeatedly. The first cycle was a series of scratches; later, the number of cycles equals the number of scratches the surface undergoes. Water contact angle and hysteresis were measured every 10 cycles (every 10 scratches or tape-peels).

### Nitric Oxide Generation

### S-Nitrosoglutathione Synthesis

S-nitrosoglutathione (GSNO) was synthesized from glutathione (GSH) with some modifications to a previously established protocol²⁴. Briefly, reduced glutathione was dissolved in dilute HCl prior to being chilled in an ice bath. Excess sodium nitrite was added to the solution and allowed to cool in the ice bath under stirring for 40 min. After, chilled acetone was added to precipitate the GSNO from the reaction. The precipitate was collected via vacuum filtration and washed with cold DI water and additional acetone. The collected filtrate was placed in a desiccator for 24 h before being ground to a fine powder with a mortar and pestle. Powder with a measured purity >95% was used and stored at −20° C., protected from light until use.

### Nitric Oxide Generation Analysis

Nitric oxide release from near physiological levels of GSNO and GSH, 1 and 30 μM respectively, in the presence of various sample types was quantified^{25,26}. Catalysis and NO generation from fabricated samples were observed in comparison to control PDMS films exposed to the same level of GSNO and GSH. To normalize and ensure the mass of metallic particles remains similar in each formulation, the weight of the films was taken prior to dip-coating and post-curing of the surface coating (Table 3, Eq. 2).

$${Mass}_{particles} ( mg ) = {[ {Mass}_{post cure} ( mg ) - {Mass}_{base PDMS} ( mg ) ]}_{*} 0.9 \qquad ( 2 )$$

TABLE 3
Weight of Particles Deposited onto Films. Assuming full evaporation
of the solvent and theoretical mass percent of PDMS in the coating
to be 10%, 90% of the difference in weight is assumed to be
metal nanoparticles and microparticles (Equation 2). Final data
for overall coating weight is reported as the mean ± standard
deviation (SD) with n ≥ 9 for each sample type.

| | Particles Deposited |
|---|---|
| Formulation | (mg cm⁻²) |
| ZnO | 6.48 ± 0.93 |
| ZnO/1Cu | 6.44 ± 1.26 |
| ZnO/5Cu | 6.45 ± 1.01 |
| ZnO/10Cu | 6.47 ± 0.74 |

Assessment of NO generation involved analysis of accumulated NO and the average NO flux using a Sievers Nitric Oxide Analyzer (280i NOA, Zysense Boulder, CO). Samples were maintained at a physiological temperature of 37° C. and protected from light. Initially, amber NOA chambers equipped with accessible ports were filled with 5994 μL of PBS and allowed to stabilize to a baseline. Following stabilization, samples were dropped into the chambers and allowed to stabilize once again. Meanwhile, a solution containing 1 mM GSNO and 30 mM GSH in PBS was prepared. A 6 μL injection of this GSNO/GSH solution was added to the low port access of the chamber using a Hamilton syringe. The resulting NO release was effectively purged from the reaction chamber using a continuous nitrogen sweep gas maintained at a constant flow rate of 200 mL min⁻¹, which was subsequently directed into a chemiluminescence reaction chamber for NO quantification. In short, NO reacts with ozone (O₃) to produce excited nitrogen dioxide (NO₂*) and oxygen. When the excited nitrogen dioxide eventually returns to its ground state, it emits a photon that can be transformed into a voltage signal and eventual value in parts per billion (ppb). The reaction is depicted in Equation (3) and Equation (4).

$$NO + {O}_{3} \to {NO}_{2}^{*} + {O}_{2} \qquad ( 3 )$$

$${NO}_{2}^{*} \to {NO}_{2} + hv \qquad ( 4 )$$

The resulting data in ppb undergoes normalization based upon the sample's surface area and the NOA's calibration constant (mol ppb⁻¹ min⁻¹). Accumulated NO measurements were determined through area-under-the-curve analysis to quantify the moles of NO released over a specific time period using MATLAB. A value for the half-life of NO in response to the final material can also be generated by observing the time at which half of the accumulated NO was achieved within the experiment. The data can also be represented in NO flux units of (×10⁻¹⁰ mol cm⁻² min⁻¹). NO recovery calculations were performed using the theoretical amount of moles released, employing simple dimensional analysis as the basis. Final data for NO generation measurements are reported as the mean±standard deviation (SD) with n≥6 for each sample type.

### Metal Leachates Analysis

To quantify metal leaching at a level comparable to the detection limits of both the Cu and Zn quantification kits, three 6 mm films were submerged in a 1.5 mL microcentrifuge tube in 1 mL of PBS for 24 h, shaking at 37° C. The copper assay detects Cu²⁺ via chromogenic chelation, producing an absorbance peak at 359 nm, while the zinc assay employs a selective fluorescent probe that increases emission upon Zn²⁺ binding (Ex/Em=485/525 nm). After 24 h, the films were removed, and the remaining PBS was used to determine the relative levels of Cu and Zn in solution.

Using a Cu colorimetric assay kit (sensitivity range of 7 μL dL⁻¹ to 300 μL dL⁻¹) and a Zn fluorometric assay kit (sensitivity of 13 ng mL⁻¹), the quantitative amount of metals identified was recorded. Both assays were run independently but used the same leachate solution. Results were normalized to the total surface area of films submerged in PBS during the incubation period. For detailed p-values and statistical significance.

### Cytocompatibility Assessment

Mouse fibroblast cells were revived from cryopreservation and maintained in complete cell culture media (DMEM, 10% FBS, 1% PS) in a 75 cm² tissue-culture treated flask. The cells were maintained at 37° C. with 5% CO₂. Routine maintenance involved detachment of the adhered cell monolayer using 0.25% (w/v) trypsin containing 0.53 mM EDTA. Following detachment, cells were washed in fresh media via centrifugation to remove residual trypsin. The washed cells were then resuspended in fresh media and either seeded into a new flask or plated for experimental use.

### NOgen Direct-Contact Cytotoxicity

For experimentation, cells were maintained as described previously. Upon resuspension in fresh complete medium, cell counting was performed using an automatic Eve cell counter (NanoEnTek, Waltham, MA). The cells were then seeded into 24-well tissue culture-treated polystyrene plates at a concentration of 50,000 cells per well. Incubation of the plates at 37° C. with 5% CO₂ overnight allowed for cell adhesion and growth until reaching approximately 80% confluency.

On the following day, all materials utilized for cytocompatibility testing were UV-sterilized, including the sample films, culture plate inserts, and 8 mL vials for subsequent use. The previous media in the plates was replaced with fresh complete media. Hanging cell culture inserts were then added to each well individually. Sterilized film samples were delicately placed into the inserts to simulate a direct leaching environment. The nature of the well inserts allowed for media to completely submerge the film inside to ensure proper mimicking of the physiological environment. NO generation was initiated by periodic injections of GSNO (1 μM) and GSH (30 μM) every hour for 4 h, with the 1 h interval selected based on the duration of NO release from the substrates in solution.

After 4 h, the study was terminated using an MTT (3-(4,5-dimethylthiazol-2-yl)-2,5-diphenyltetrazolium bromide) colorimetric assay. The films, inserts, and media were removed from each well and the remaining cell monolayers were supplemented with 0.5 mg mL⁻¹ of MTT reagent. This reagent permeated the cells and reacted with any active cellular metabolisms, resulting in the formation of purple formazan crystals over 1-2 h at 37° C. with 5% CO₂. These crystals were then dissolved using dimethyl sulfoxide (DMSO) to generate a gradient of purple solutions, which were analyzed using a Biotek Cytation™ 5 Cell-Imaging Multi-Mode Reader (Winooski, VT). Final cell viability was determined by comparing absorbance readings from the experimental groups against control wells that were not treated (Equation (5)).

$$Relative Cell Viability ( \% ) = \frac{{( {OD}_{450} - {OD}_{650} )}_{Test group}}{{( {OD}_{450} - {OD}_{650} )}_{Control group}} \times 100$$

### Antibacterial Assessment

The combination strategies of the final material aim to reduce planktonic bacterial counts, prevent bacterial adhesion, and induce bacterial killing should adhesion occur.

To quantify bacterial reductions, a single S. aureus colony was isolated and inoculated into LB media and grown at 37° C. under orbital shaking (150 RPM) until reaching the log phase of growth. The suspension was then centrifuged at 4400 RPM for 7 min to yield a bacterial pellet. LB media was decanted, and the bacterial pellet was resuspended in sterile PBS to 0.1 OD₆₀₀, corresponding to ~10⁸ CFU mL⁻¹.

### Vascular Mimicking Bacterial Adhesion Assessment

For each adhesion assessment, samples were evaluated under two conditions (n=4 per condition): (i) films without GSNO/GSH [Control (−), ZnO/10Cu (−)] and (ii) corresponding films supplemented with GSNO (1 μM) and GSH (30 μM) [Control (+), ZnO/10Cu (+)], for a total of n=8 per sample type.

Each sample was exposed to 1 mL of 0.1 OD₆₀₀ bacterial solution for a total of 4 h, with respective injections every 1 h (4 injections total). After 4 h incubation, samples were taken out of bacterial suspension and rinsed 3 times in sterile PBS to remove any loosely adhered bacteria. Films were then placed in 1 mL of sterile PBS and were homogenized to remove any adhered bacteria. Homogenized solutions were diluted to provide countable colony counts and plated onto LB agar using an Eddy Jet W2 spiral plater (IUL, Farmingdale, NY). Once the dilutions were spiral plated, LB agar petri dishes were placed upside down and incubated overnight at 37° C. to allow viable colonies to grow. Colonies were counted and subsequently analyzed with a SphereFlash automated colony counter (IUL, Farmingdale, NY). All sample types were plated undiluted (neat) or diluted at 1:100 in sterile PBS (2^{nd} dilution).

### Vascular Mimicking Planktonic Killing Assessment

Similar to adhesion assessments, each film was exposed to 1 mL of 0.1 OD₆₀₀ bacterial solution for 4 h with the identical injection schedule. After 4 h incubation, samples were taken out of bacterial suspension. The remaining bacterial solutions were diluted to provide countable colony counts and plated onto LB agar and treated identically to adhesion study conditions. All sample types were plated at ratios of 1:100 (2^{nd} dilution) or 1:10000 (4^{th} dilution) in sterile PBS. For detailed p-values and statistical significance.

### Statistical Analyses

All data is reported as mean±standard deviation unless otherwise specified. Statistical significance between sample types was obtained from performing ordinary one-way analysis of variance (ANOVA) tests. Comparisons with a p-value of <0.05 were deemed significant. Any significance data not displayed can be found in supplementary information.

### Results and Discussion

### Synthesis and Surface Characterization

A lotus leaf-inspired non-adhesive superhydrophobic coating was achieved through the incorporation of silanized ZnO and Cu particles within a siloxane matrix. Superhydrophobic performance is characterized by static water contact angles (WCA) greater than 150° and water contact angle hysteresis (CAH) below 10°.^{12,27-31} These properties arise from the combined effects of micro- and nanoscale roughness and low-surface-energy chemistry. In this work, hydrophilic metal and metal oxide particles were modified with octadecyltrimethoxysilane (ODTMS), a long-chain silane, to impart low surface energy. The viscosity of the metal/metal-oxide-ODTMS suspension was then adjusted to a paint-like consistency and incorporated within a polydimethylsiloxane (PDMS) base to improve adhesion of the coating to underlying PDMS substrates.

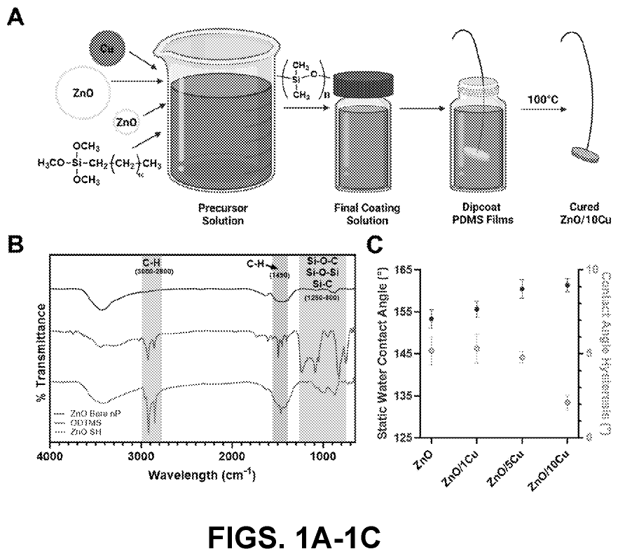

The three-step fabrication process for creating the superhydrophobic, NO-generating surface is illustrated in FIG. 1A. A precursor solution containing ZnO nanoparticles and microparticles, Cu nanoparticles, and ODTMS in hexane was first prepared. The use of ZnO microparticles and nanoparticles, together with intermediate-sized Cu nanoparticles, was intentionally selected to establish a hierarchical distribution of feature sizes across multiple scale lengths. Sylgard-184 PDMS was subsequently incorporated at 10 wt %, and the resulting mixture was applied to PDMS films via dip-coating and cured to form superhydrophobic surfaces. Bare Sylgard-184 PDMS films served as controls for performance evaluation.

The silanization of the metal/metal oxide particles by ODTMS was hypothesized to embed superhydrophobic properties. ODTMS, a long-chain aliphatic silane, was selected as a non-fluorinated, low-surface-energy modifier to reduce potential toxicity concerns associated with fluorinated silanes.³²⁻³⁴ To verify this modification, unmodified and silanized metallic particles derived from the precursor solution were analyzed by FTIR (FIG. 1B). Modified ZnO particles are denoted as ZnO SH to distinguish them from unmodified ZnO. In addition to spectroscopic confirmation, covalent silanization was further supported by a change in particle wettability from hydrophilic to superhydrophobic.

Based on the molecular structure of ODTMS, FTIR spectra were expected to exhibit characteristic vibrations associated with C—H stretching, Si—O—C, Si—O—Si, and Si—C bonds. As shown in FIG. 1B, neat ODTMS displayed prominent peaks between 2800-3000 cm⁻¹ and near 1490 cm⁻¹, corresponding to C—H stretching and bending of the aliphatic chains. Additional peaks near 1250 cm⁻¹, 1100 cm⁻¹, and within the 800-1000 cm⁻¹ region were assigned to Si—O—C, Si—O—Si, and Si—C stretching vibrations, respectively.

When compared to unmodified ZnO, silanized ZnO (ZnO SH) exhibited pronounced peaks in the 2800-3000 cm⁻¹ and 1490 cm⁻¹ regions, along with increased intensity within the 900-1100 cm⁻¹ range, indicating successful grafting of ODTMS onto the particle surface. Collectively, these spectral features confirm effective silanization, which is necessary to impart low surface energy and enable subsequent formation of non-adhesive superhydrophobic coatings.

Hierarchical micro/nanoscale surface architecture has been widely reported as essential for achieving durable superhydrophobicity. Previous studies have demonstrated that micro- or nano-structured surfaces alone fail to exhibit stable superhydrophobic behavior, whereas hierarchical structures enhance wettability by stabilizing the trapped air layer and improving resistance to wetting transitions.^{35,36} Consistent with these principles, varying ratios of ZnO nanoparticles and microparticles were initially explored to assess general coating robustness. To minimize experimental values and enable direct comparison among formulations, a 50:50 ratio of ZnO nanoparticles to microparticles was selected. This ratio was used for all subsequent studies.

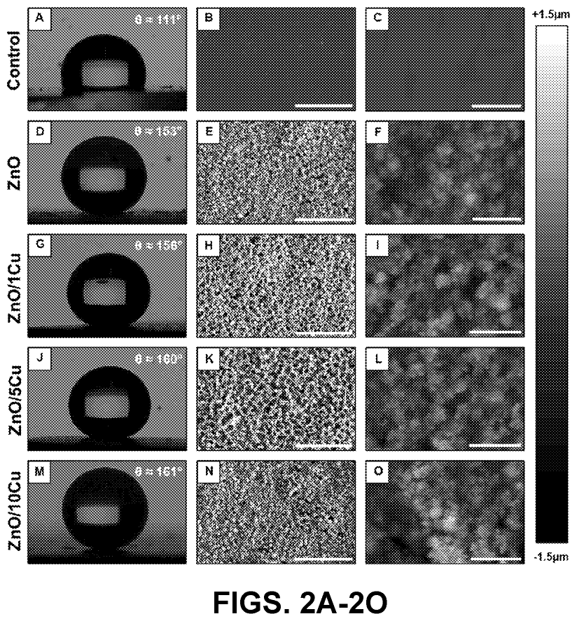

All fabricated coatings exhibited static water contact angles (WCA) exceeding 150°, in contrast to control PDMS, which displayed an average WCA of 111° (FIGS. 1C, 2A-O). In addition, all superhydrophobic samples demonstrated contact angle hysteresis (CAH) values well below 10°, confirming non-adhesive superhydrophobic behavior.

As Cu content increased, WCA increased and CAH decreased. This trend is attributed to enhanced hierarchical structuring arising from the introduction of Cu nanoparticles (40-60 nm), which provide intermediate feature sizes between ZnO nanoparticles (10-30 nm) and ZnO microparticles (<5 μm). The distribution of particles across micro- and nanoscale ranges promotes stabilization of the trapped air layer and improves resistance to wetting.

Surface morphology and coating uniformity were further examined by SEM, EDS, and AFM (FIGS. 2A-O). All coated samples exhibited textures distinctly different from bare PDMS, confirming successful deposition of the particle-based coating. No pronounced morphological differences were observed among ZnO, ZnO/1Cu, ZnO/5Cu, and ZnO/10Cu by SEM. Elemental mapping revealed uniform distributions of Zn and Cu across the coating surface, with quantitative elemental compositions. These results indicate consistent incorporation of both metal and metal oxide particles throughout the coating.

AFM analysis revealed a progressive increase in root-mean-square roughness (Rq) with increasing Cu content, from 19.58±5.31 nm for control films to 177.25±23.46 nm for ZnO, 236±22.66 nm for ZnO/1Cu, 288±9.09 nm for ZnO/5Cu, and 353.75±26.04 nm for ZnO/10Cu. All coated samples were significantly rougher than the control. The monotonic increase in roughness with Cu incorporation is consistent with the observed improvements in WCA and CAH, further supporting the role of hierarchical surface architecture in governing superhydrophobic performance.

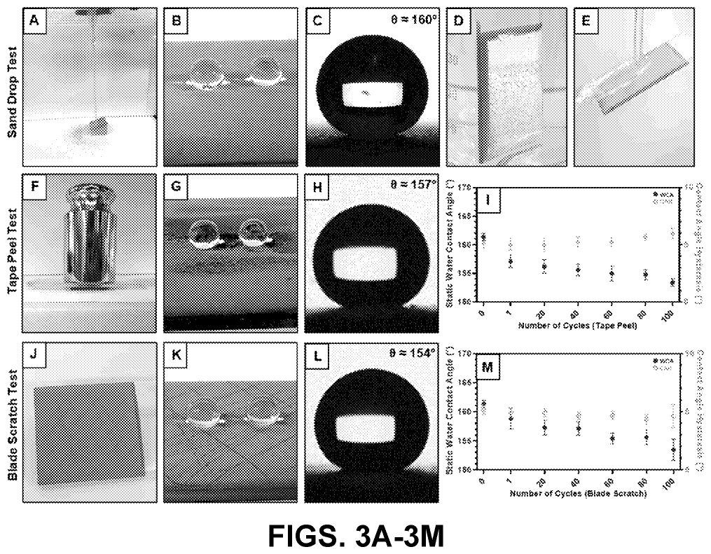

The mechanical durability of the ZnO/10Cu coating was evaluated to assess the stability of the embedded non-adhesive superhydrophobicity under representative mechanical and aqueous stress conditions (FIGS. 3A-3M). Following a sand-drop test in which 10 g of sand was dropped from a height of 20 cm, the ZnO/10Cu surface maintained a WCA of 159°±1.5, indicating minimal disruption of superhydrophobic behavior (FIGS. 3A-C). When submerged in deionized water, the ZnO/10Cu surface exhibited a visible air plastron layer (FIG. 3D), confirming the presence of a trapped air layer within the surface texture. Under continuous water-jet exposure, the coating retained its integrity and water-repellent behavior, with droplets exhibiting pronounced beading and rapid roll-off (FIG. 3E).

Adhesive tape peeling and blade scratching tests were conducted to further evaluate mechanical robustness. During 100 cycles of tape peeling, WCA remained above 150° and CAH remained below 10°, despite a slight decrease in WCA after the initial cycle (FIGS. 3F-I). Similarly, after repeated blade-scratching, WCA remained above 150° and CAH remained below 10° (FIGS. 3J-M). Collectively, these results demonstrate that the ZnO/10Cu coating retains superhydrophobic performance under mechanical abrasion and in aqueous conditions. Although a modest decrease in WCA was observed following repeated scratching, the coating retained highly water-repellent behavior and exhibited strong resistance to mechanical abrasion.

### Investigation of Nitric Oxide Generation

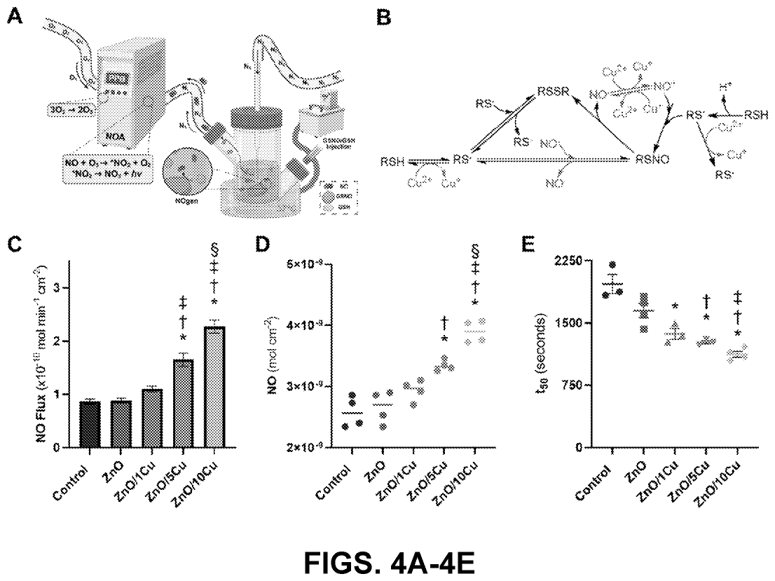

The ability of the metal-based superhydrophobic coatings to catalytically generate NO from physiological RSNOs was investigated under low-end physiological GSNO/GSH concentrations. All samples, including the PDMS control, were exposed to identical injections of GSNO and GSH to ensure that differences in NO release reflected catalytic activity at the material surface rather than variations in substrate availability (FIG. 4A). Catalytic NO generation is sensitive to the availability of redox-active metal species, making consistent particle loading across samples critical for meaningful comparison of NO-generation performance.

To ensure uniformity, the mass of particles deposited on each substrate was quantified after excluding contributions from the PDMS substrate and solvent. No significant differences in particle loading were observed among sample types, indicating that variations in ZnO:Cu ratios did not introduce systematic differences in coating mass. These results enable direct comparison of catalytic activity and NO-generation behavior across formulations.

Catalytic generation of NO from RSNOs by metal-based materials has been widely reported, particularly for copper-containing systems in which Cu²⁺ is reduced to Cu⁺ by thiolates or other physiological reducing agents, followed by Cu⁺-mediated decomposition of GSNO to release NO (FIG. 4B).²⁰⁻²² This Cu-mediated RSNO decomposition pathway has been well established in NO-generating biomaterials, including previous studies from our group demonstrating copper-driven catalytic NO generation from physiological S-nitrosothiols.^{37,38} Similar redox-active behavior has also been reported for other transition metals, including iron, nickel, cobalt, manganese, and zinc, depending on oxidation state and coordination environment.^{23,39} In the context of these established mechanisms, the NO-generation performance of the fabricated coatings was systematically evaluated. Three key metrics were examined: average peak NO flux, total accumulated NO normalized to surface area, and tso, defined as the time required for 50% of the total NO load to be released following exposure to GSNO/GSH.

Average peak NO flux increased with increasing Cu content in the coatings, with ZnO/10Cu exhibiting the highest peak flux (2.28±0.25 x10⁻¹⁰ mol cm⁻² min⁻¹), followed by ZnO/5Cu (1.65±0.22 x10⁻¹⁰ mol cm⁻² min⁻¹), ZnO/1Cu (1.10±0.10 x10⁻¹⁰ mol cm⁻² min⁻¹), and ZnO (0.88±0.09 x10⁻¹⁰ mol cm⁻² min⁻¹), while the control PDMS displayed the lowest NO flux (0.86±0.08 x10⁻¹⁰ mol cm⁻² min⁻¹)(FIG. 4C). Peak NO flux primarily reflects the rate at which RSNOs are catalytically decomposed at the material surface. Accordingly, the observed increase in peak flux with Cu incorporation indicates enhanced catalytic activity arising from a greater density of redox-active Cu sites capable of facilitating the Cu²⁺/Cu⁺ redox cycle. Importantly, peak flux alone does not describe the total amount of NO generated, necessitating evaluation of accumulated NO to assess true NO-generation capacity.

Total accumulated NO, normalized to surface area, also increased with Cu content, with ZnO/10Cu generating the highest accumulated NO among all sample types and exhibiting an 86.40±4.0% increase in NO generated relative to the PDMS control (FIG. 4D). In absolute terms, ZnO/10Cu produced 3.90±0.18 nmol cm⁻² accumulated NO and significantly exceeded ZnO/5Cu (3.34±0.09 nmol cm⁻²). ZnO/5Cu and ZnO/1Cu produced greater accumulated NO than ZnO and the control, while ZnO displayed only a slight, non-significant increase relative to the control. The limited NO generation observed for ZnO alone likely reflects the absence of efficient redox cycling at the ZnO surface. Metal oxides such as ZnO possess relatively stable oxidized metal centers and higher redox potentials compared to their metallic counterparts, which can limit participation in catalytic redox cycling.^{39,40} While ZnO can participate in surface interactions with thiols, it lacks the readily accessible redox couple required to efficiently catalyze RSNO decomposition. In contrast, incorporation of Cu introduces redox-active sites capable of cycling between Cu²⁺ and Cu⁺, which facilitates sustained catalytic decomposition of GSNO and subsequent NO release.

To further probe the influence of Cu content, coatings containing Cu concentrations above 10 wt % were examined, yielding a ZnO/20Cu formulation. However, accumulated NO generated from ZnO/20Cu was not significantly different from that of ZnO/10Cu, indicating that 10 wt % Cu represents a practical upper limit for maximizing NO-generation capacity within this system.

Kinetic persistence of NO generation was evaluated using t₅₀, defined as the time required for 50% of the total NO to be released following exposure to GSNO/GSH (FIG. 4E). The t₅₀ decreased with increasing Cu content, with ZnO/10Cu exhibiting a t₅₀ of 1123±70 s compared to 1970±200 s for the control. This reduction in t₅₀ further supports a Cu-mediated catalytic mechanism, in which higher densities of redox-active sites accelerate RSNO decomposition and NO release.

Collectively, these results demonstrate that Cu incorporation not only accelerates NO-generation kinetics but also increases overall NO yield. The distinction between increased peak flux (reflecting catalytic activity) and increased accumulated NO (reflecting total generation capacity) confirms that ZnO/10Cu functions as an effective NO-generating surface. With ZNO/10Cu exhibiting the highest peak flux, the highest accumulated NO, and the shortest t₅₀ among the tested formulations, this composition was selected for subsequent biological studies. However, given the potential toxicity of metal-containing materials, comprehensive metal-leaching studies and cytocompatibility assessments were performed prior to antibacterial evaluation.

### Evaluation of Metal Leaching

Given the incorporation of metal and metal oxide particles within the coating, quantitative assessment of Zn and Cu leaching was performed to evaluate material stability and potential toxicity. Samples were incubated in PBS for 24 h under conditions described in the Methods, after which Zn and Cu concentrations in the leachates were quantified using metal detection kits.

Low levels of Zn and Cu were detected for all sample types (Table 4). ZnO exhibited minimal Zn release, and incorporation of Cu did not increase Zn leaching relative to ZnO alone. For Cu-containing formulations, measurable Cu leaching was observed; however, the detected concentrations were substantially below toxic levels reported in literature.⁴¹ As expected, the mass of leached Cu increased with increasing Cu content, though the percentage of Cu released relative to the amount deposited onto each film remained below 0.1% for all formulations.

TABLE 4
Zinc and Copper Metal Leaching

| | Cu Leachates | Zn Leachates | Cu Leached* | Zn Leached* |
|---|---|---|---|---|
| Formulation | (μg cm⁻²) | (μg cm⁻²) | (%) | (%) |
| ZnO | — | 0.073 ± 0.013 | — | 0.0015 ± 0.0003 |
| ZnO/1Cu | 0.142 ± 0.034 | 0.063 ± 0.007 | 0.029 ± 0.070 | 0.0013 ± 0.0001 |
| ZnO/5Cu | 0.166 ± 0.023 | 0.062 ± 0.011 | 0.072 ± 0.010 | 0.0013 ± 0.0002 |
| ZnO/10Cu | 0.437 ± 0.014 | 0.059 ± 0.001 | 0.096 ± 0.003 | 0.0013 ± 0.0003 |

*Relative to the amount of Cu and Zn in the coating solutions and the amount deposited onto each film

These results indicate strong retention of both Zn and Cu within the coating and suggest that NO-generation behavior arises primarily from surface-mediated catalytic activity rather than passive metal ion release. The low extent of metal leaching further supports the suitability of these coatings for subsequent biological evaluation.

### Cytocompatibility Assessment

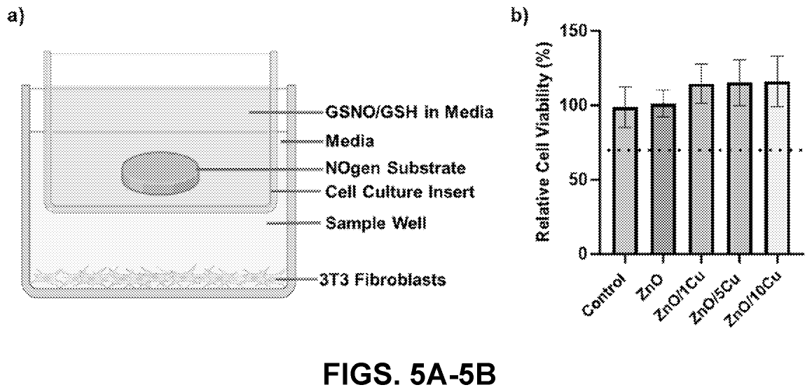

Cytocompatibility of the fabricated coatings was evaluated using a direct-leaching model with 3T3 mouse fibroblasts. Cells were seeded and cultured to approximately 80% confluency, after which hanging cell culture inserts were placed into each well, and samples were introduced within the inserts to simulate a direct exposure environment (FIG. 5A). To establish NO-generating conditions, GSNO and GSH were injected into each well at final concentrations of 1 μM and 30 μM, respectively, with injections repeated hourly over the 4 h experiment.

Following 4 h of exposure, relative cell viability was quantified using an MTT assay and compared to cells exposed to inserts without material or GSNO/GSH treatment. All sample types exhibited cell viabilities greater than 70%, meeting ISO cytocompatibility criteria (FIG. 5B).⁴²

These results, in conjunction with the low levels of Zn and Cu leaching, demonstrate that the coatings maintain cytocompatibility while supporting catalytic NO generation under physiologically relevant conditions. Importantly, the preservation of fibroblast viability suggests that NO produced at these low-end physiological RSNO/GSH concentrations does not induce acute cytotoxicity. Notably, the transwell-based exposure model was intentionally designed to assess cytocompatibility of the coating under vascular-mimicking NO-generating conditions, in which endogenous RSNOs are continuously converted to NO at the material interface. Together, these findings indicate that incorporation of redox-active metals within the superhydrophobic matrix enables functional NO generation without compromising cellular compatibility, supporting the suitability of these coatings for subsequent antibacterial evaluation and potential biomedical applications.

### Antibacterial Assessment

Antibacterial performance of the optimized ZnO/10Cu coating was evaluated using a purpose-designed, vascular-mimicking NO-generating model that integrates physiological RSNO/GSH delivery with surface-mediated catalytic NO-generation. This model was developed to assess antibacterial behavior under conditions that more closely approximate physiologically relevant NO-rich microenvironments. To establish low-end physiological NO-generating conditions, GSNO and GSH were injected hourly to achieve final concentrations of 1 μM and 30 μM, respectively. PDMS, ZnO, and ZnO/10Cu samples were evaluated under untreated (PBS only) and treated (GSNO/GSH) conditions to decouple the effects of substrate chemistry from those of the NO-generating environment. In this system, ZnO serves as an intermediate material that provides superhydrophobic surface roughness and modest antibacterial behavior, whereas incorporation of Cu enables catalytic NO-generation from physiological levels of RSNOs.

The ZnO/10Cu coating exhibits both non-adhesive superhydrophobicity and catalytic NO-generation; therefore, antibacterial performance was evaluated in terms of (i) bacterial addition to the surface and (ii) planktonic bacterial viability in the surrounding medium. Evaluation of planktonic bacteria is particularly important, as many superhydrophobic surfaces primarily report reductions in surface-associated bacteria rather than bactericidal activity. Quantifying planktonic bacteria in the surrounding medium, therefore, enables the determination of whether additional antimicrobial effects arise from surface-mediated NO generation beyond any intrinsic antibacterial contributions of GSNO/GSH alone.

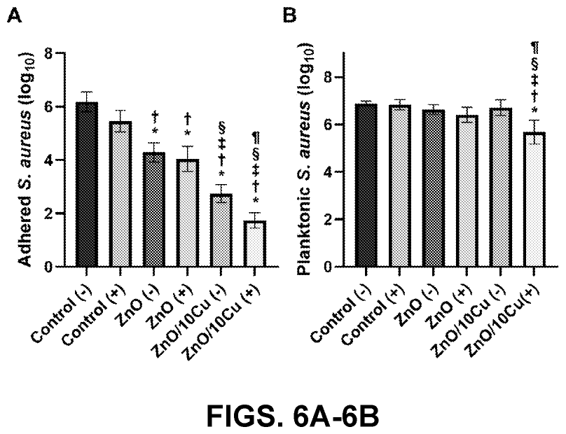

ZnO/10Cu surfaces exhibited significantly fewer adhered S. aureus compared to both PDMS and ZnO controls under both untreated and GSNO/GSH-treated conditions (FIG. 6A). Relative to untreated PDMS, ZnO/10Cu demonstrated a 3.45-log reduction in adhered bacteria, while a 4.45-log reduction was observed under NO-generating conditions. ZnO surfaces also showed reduced bacterial adhesion compared to PDMS, consistent with the anti-adhesive behavior of superhydrophobic surfaces and the modest antibacterial activity reported for ZnO-containing coatings. However, adhered bacterial counts on ZnO remained significantly higher than those observed for ZnO/10Cu.

Only minor, non-significant differences were observed between untreated and GSNO/GSH-treated PDMS and ZnO samples, indicating minimal antibacterial contribution from GSNO/GSH alone at these concentrations. In contrast, a statistically significant ~1.0-log reduction was observed between untreated and treated ZnO/10Cu samples. These results indicate that no-adhesive superhydrophobicity is the dominant contributor to reduced bacterial attachment, whereas surface-mediated NO-generation provides an additional antibacterial effect even at low physiological GSNO/GSH concentrations, further suppressing viable adhered bacteria.

Planktonic S. aureus viability was quantified following the 4 h exposure to evaluate whether surface-mediated NO-generation impacts bacteria in the surrounding medium in addition to reducing surface adhesion (FIG. 6B). No significant differences in planktonic bacterial counts were observed among untreated PDMS, treated PDMS, untreated ZnO, treated ZnO, or untreated ZnO/10Cu samples. In contrast, ZnO/10Cu under NO-generating conditions exhibited a significant reduction in planktonic bacteria, corresponding to approximately a 1.2-log decrease relative to untreated control PDMS.

These results demonstrate that GSNO/GSH alone at the tested concentrations does not appreciably reduce planktonic bacteria and that metal leaching from ZnO/10Cu is not responsible for bactericidal activity in solution. Rather, the significant planktonic killing observed exclusively for ZnO/10Cu under NO-generating conditions provides direct functional evidence of catalytic NO-generation, confirming that NO produced at the material interface diffuses into the surrounding medium and exerts bactericidal activity. This represents a critical validation of true NO-generating behavior, extending beyond chemical detection alone to demonstrate biological efficacy.

Collectively, these findings demonstrate that the ZnO/10Cu coating integrates non-adhesive superhydrophobicity with catalytic NO-generation to achieve robust antibacterial performance. Superhydrophobic surface features substantially limit initial bacterial attachment, whereas surface-mediated NO-generation provides additional suppression of both adhered and planktonic bacteria under vascular-mimicking conditions. The ability to simultaneously reduce surface-associated bacteria and inactivate microorganisms in the surrounding medium highlights the advantage of combining physical antibiofouling with localized, catalytic antimicrobial functionality within a single coating platform.

### CONCLUSION

A metal-siloxane-derived, non-adhesive superhydrophobic coating capable of nitric oxide (NO) generation from physiological S-nitrosothiols was developed and systematically evaluated. Unlike prior approaches that rely on cytotoxic fluorosilanes, the coating employs ZnO and Cu particles and ODTMS to achieve hierarchical micro/nanoscale roughness, low surface energy, and durable superhydrophobicity. Incorporation of Cu enables surface-mediated NO generation under low-end physiological RSNO/GSH conditions, and kinetic and cumulative NO analyses confirmed that Cu incorporation enhances both NO-generation rate and total NO output, with ZnO/10Cu identified as an optimized formulation.

Low levels of Zn and Cu leaching and high fibroblast viability (>70%) indicate that functional performance is achieved without compromising cytocompatibility. Using a vascular-mimicking NO-generating model, the coating substantially reduced bacterial adhesion (99.99%) and significantly decreased planktonic bacterial viability (89.43%), representing one of the first demonstrations of successful antibacterial performance achieved through catalytic NO generation from endogenous RSNOs. These results provide direct biological validation of true NO-generating behavior.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

(1) Healthcare-Associated Infections (HAIs). Centers for Disease Control and Prevention (CDC), https://www.cdc.gov/healthcare-associated-infections/php/data/progress-report.html (accessed 2025 August 15).
(2) Lichter, J. A.; Van Vliet, K. J.; Rubner, M. F. Design of antibacterial surfaces and interfaces: polyelectrolyte multilayers as a multifunctional platform. Macromolecules 2009, 42 (22), 8573-8586.
(3) Donlan, R. M. Biofilms and device-associated infections. Emerging infectious diseases 2001, 7(2), 277.
(4) Harding, J. L.; Reynolds, M. M. Combating medical device fouling. Trends in biotechnology 2014, 32 (3), 140-146.
(5) Lieberman, J. M. Appropriate antibiotic use and why it is important: the challenges of bacterial resistance. The Pediatric infectious disease journal 2003, 22 (12), 1143-1151.
(6) Francolini, I.; Vuotto, C.; Piozzi, A.; Donelli, G. Antifouling and antimicrobial biomaterials: an overview. Apmis 2017, 125 (4), 392-417.
(7) Ozkan, E.; Bright, L. M. E.; Kumar, A.; Pandey, R.; Devine, R.; Francis, D.; Ghalei, S.; Ashcraft, M.; Maffe, P.; Brooks, M. Bioinspired superhydrophobic surfaces with silver and nitric oxide-releasing capabilities to prevent device-associated infections and thrombosis. Journal of Colloid and Interface Science 2024, 664, 928-937.
(8) Ozkan, E.; Mondal, A.; Douglass, M.; Hopkins, S. P.; Garren, M.; Devine, R.; Pandey, R.; Manuel, J.; Singha, P.; Warnock, J. Bioinspired ultra-low fouling coatings on medical devices to prevent device-associated infections and thrombosis. Journal of colloid and interface science 2022, 608, 1015-1024.
(9) Ozkan, E.; Mondal, A.; Singha, P.; Douglass, M.; Hopkins, S. P.; Devine, R.; Garren, M.; Manuel, J.; Warnock, J.; Handa, H. Fabrication of bacteria- and blood-repellent superhydrophobic polyurethane sponge materials. ACS applied materials & interfaces 2020, 12 (46), 51160-51173.
(10) Ozkan, E.; Garren, M.; Manuel, J.; Douglass, M.; Devine, R.; Mondal, A.; Kumar, A.; Ashcraft, M.; Pandey, R.; Handa, H. Superhydrophobic and conductive foams with antifouling and oil-water separation properties. ACS Applied Materials & Interfaces 2023, 15 (5), 7610-7626.
(11) Leslie, D. C.; Waterhouse, A.; Berthet, J. B.; Valentin, T. M.; Watters, A. L.; Jain, A.; Kim, P.; Hatton, B. D.; Nedder, A.; Donovan, K. A bioinspired omniphobic surface coating on medical devices prevents thrombosis and biofouling. Nature biotechnology 2014, 32 (11), 1134-1140.
(12) Cassie, A.; Baxter, S. Wettability of porous surfaces. Transactions of the Faraday society 1944, 40, 546-551.
(13) Ozkan, E.; Estes Bright, L. M.; Kumar, A.; Pandey, R.; Devine, R.; Francis, D.; Ghalei, S.; Ashcraft, M.; Maffe, P.; Brooks, M.; et al. Bioinspired superhydrophobic surfaces with silver and nitric oxide-releasing capabilities to prevent device-associated infections and thrombosis. Journal of Colloid and Interface Science 2024, 664, 928-937. DOI: https://doi.org/10.1016/j.jcis.2024.03.082.
(14) Ozkan, E.; Mondal, A.; Douglass, M.; Hopkins, S. P.; Garren, M.; Devine, R.; Pandey, R.; Manuel, J.; Singha, P.; Warnock, J.; Handa, H. Bioinspired ultra-low fouling coatings on medical devices to prevent device-associated infections and thrombosis. Journal of Colloid and Interface Science 2022, 608, 1015-1024. DOI: https://doi.org/10.1016/j.jcis.2021.09.183.
(15) Bogdan, C.; Röllinghoff, M.; Diefenbach, A. The role of nitric oxide in innate immunity. Immunol Rev 2000, 173, 17-26. DOI: 10.1034/j.1600-065x.2000.917307.x From NLM.
(16) Mendhi, J.; Asgari, M.; Ratheesh, G.; Prasadam, I.; Yang, Y.; Xiao, Y. Dose controlled nitric oxide-based strategies for antibacterial property in biomedical devices. Applied Materials Today 2020, 19, 100562.
(17) Fang, F. C. Perspectives series: host/pathogen interactions. Mechanisms of nitric oxide-related antimicrobial activity. The Journal of clinical investigation 1997, 99 (12), 2818-2825.
(18) Arora, D. P.; Hossain, S.; Xu, Y.; Boon, E. M. Nitric oxide regulation of bacterial biofilms. Biochemistry 2015, 54 (24), 3717-3728.
(19) Askew, S. C.; Barnett, D. J.; McAninly, J.; Williams, D. L. H. Catalysis by Cu2+ of nitric oxide release from S-nitrosothiols (RSNO). Journal of the Chemical Society-Perkin Transactions II 1995, (4), 741-746.
(20) Dicks, A.; áHerves Beloso, P. Decomposition of S-nitrosothiols: the effects of added thiols. Journal of the Chemical Society, Perkin Transactions 2 1997, (8), 1429-1434.
(21) Gorren, A. C.; Schrammel, A.; Schmidt, K.; Mayer, B. Decomposition of S-Nitrosoglutathione in the Presence of Copper Ions and Glutathione. Archives of Biochemistry and Biophysics 1996, 330 (2), 219-228.
(22) Lyn H áWilliams, D. Identification of Cu+ as the effective reagent in nitric oxide formation from S-nitrosothiols (RSNO). Journal of the Chemical Society, Perkin Transactions 2 1996, (4), 481-487.
(23) McCarthy, C. W.; Guillory, R. J., II; Goldman, J.; Frost, M. C. Transition-Metal-Mediated Release of Nitric Oxide (NO) from S-Nitroso-N-acetyl-d-penicillamine (SNAP): Potential Applications for Endogenous Release of NO at the Surface of Stents Via Corrosion Products. ACS Applied Materials & Interfaces 2016, 8 (16), 10128-10135. DOI: 10.1021/acsami.6b00145.
(24) Hart, T. W. Some observations concerning the S-nitroso and S-phenylsulphonyl derivatives of L-cysteine and glutathione. Tetrahedron Letters 1985, 26 (16), 2013-2016.
(25) Wu, Y.; Rojas, A. P.; Griffith, G. W.; Skrzypchak, A. M.; Lafayette, N.; Bartlett, R. H.; Meyerhoff, M. E. Improving blood compatibility of intravascular oxygen sensors via catalytic decomposition of S-nitrosothiols to generate nitric oxide in situ. Sensors and Actuators B: Chemical 2007, 121 (1), 36-46. DOI: https://doi.org/10.1016/j.snb.2006.09.025.
(26) Giustarini, D.; Milzani, A.; Colombo, R.; Dalle-Donne, I.; Rossi, R. Nitric oxide and S-nitrosothiols in human blood. Clinica Chimica Acta 2003, 330 (1), 85-98. DOI: https://doi.org/10.1016/S0009-8981(03)00046-9.
(27) Feng, L.; Li, S.; Li, Y.; Li, H.; Zhang, L.; Zhai, J.; Song, Y.; Liu, B.; Jiang, L.; Zhu, D. Super-hydrophobic surfaces: from natural to artificial. Advanced materials 2002, 14 (24), 1857-1860.
(28) Gao, L.; McCarthy, T. J. The “lotus effect” explained: two reasons why two length scales of topography are important. Langmuir 2006, 22 (7), 2966-2967.
(29) Nishimura, R.; Hyodo, K.; Sawaguchi, H.; Yamamoto, Y.; Nonomura, Y.; Mayama, H.; Yokojima, S.; Nakamura, S.; Uchida, K. Fractal surfaces of molecular crystals mimicking lotus leaf with phototunable double roughness structures. Journal of the American Chemical Society 2016, 138 (32), 10299-10303.
(30) Wang, S. Definition of superhydrophobic states. Advanced materials 2007, 19 (21), 3423-3424.
(31) Barthlott, W.; Neinhuis, C. Purity of the sacred lotus, or escape from contamination in biological surfaces. Planta 1997, 202, 1-8.
(32) Daly, E. R.; Chan, B. P.; Talbot, E. A.; Nassif, J.; Bean, C.; Cavallo, S. J.; Metcalf, E.; Simone, K.; Woolf, A. D. Per- and polyfluoroalkyl substance (PFAS) exposure assessment in a community exposed to contaminated drinking water, New Hampshire, 2015. International journal of hygiene and environmental health 2018, 221 (3), 569-577.
(33) Guardian, M. G. E.; Boongaling, E. G.; Bernardo-Boongaling, V. R. R.; Gamonchuang, J.; Boontongto, T.; Burakham, R.; Arnnok, P.; Aga, D. S. Prevalence of per- and polyfluoroalkyl substances (PFASs) in drinking and source water from two Asian countries. Chemosphere 2020, 256, 127115.
(34) EPA, U. PFAS strategic roadmap: EPA's commitments to action 2021-2024. Environmental Protection Agency: Washington, DC 2021, 20460.
(35) Wang, Q.; Xu, S.; Xing, X.; Wang, N. Progress in fabrication and applications of micro/nanostructured superhydrophobic surfaces. Surface Innovations 2021, 10 (2), 89-110.
(36) Koch, K.; Bhushan, B.; Jung, Y. C.; Barthlott, W. Fabrication of artificial Lotus leaves and significance of hierarchical structure for superhydrophobicity and low adhesion. Soft Matter 2009, 5 (7), 1386-1393.
(37) Douglass, M. E.; Goudie, M. J.; Pant, J.; Singha, P.; Hopkins, S.; Devine, R.; Schmiedt, C. W.; Handa, H. Catalyzed nitric oxide release via Cu nanoparticles leads to an increase in antimicrobial effects and hemocompatibility for short-term extracorporeal circulation. ACS Applied Bio Materials 2019, 2 (6), 2539-2548.
(38) Pant, J.; Goudie, M. J.; Hopkins, S. P.; Brisbois, E. J.; Handa, H. Tunable nitric oxide release from S-nitroso-N-acetylpenicillamine via catalytic copper nanoparticles for biomedical applications. ACS applied materials & interfaces 2017, 9 (18), 15254-15264.
(39) Kuznetsov, D. A.; Han, B.; Yu, Y.; Rao, R. R.; Hwang, J.; Román-Leshkov, Y.; Shao-Horn, Y. Tuning redox transitions via inductive effect in metal oxides and complexes, and implications in oxygen electrocatalysis. Joule 2018, 2 (2), 225-244.
(40) Reddy, B. M. Redox Properties of Metal Oxides. ChemInform 2006, 37 (10). DOI: https://doi.org/10.1002/chin.200610244.
(41) Song, L.; Connolly, M.; Fernández-Cruz, M. L.; Vijver, M. G.; Fernández, M.; Conde, E.; de Snoo, G. R.; Peijnenburg, W. J.; Navas, J. M. Species-specific toxicity of copper nanoparticles among mammalian and piscine cell lines. Nanotoxicology 2014, 8 (4), 383-393.
(42) Standard, I. Biological evaluation of medical devices—Part 5: Tests for in vitro cytotoxicity. Geneve, Switzerland: International Organization for Standardization 2009.

1. A composition comprising
(a) metal oxide nanoparticles;
(b) metal oxide microparticles;
(c) metal particles selected from the group consisting of silver, copper, strontium, selenium, zinc, iron, nickel, and any combination thereof; and
(d) a silane compound.

2. The composition of claim 1, wherein the metal oxide nanoparticles comprise zinc oxide, silicon oxide, aluminum oxide, titanium oxide, iron oxide, or any combination thereof.

3. The composition of claim 1, wherein the metal oxide microparticles comprise zinc oxide, silicon oxide, aluminum oxide, titanium oxide, iron oxide, or any combination thereof.

4. The composition of claim 1, wherein the metal oxide nanoparticles have an average particle size of from about 5 nm to about 100 nm.

5. The composition of claim 1, wherein the metal oxide nanoparticles and the metal oxide nanoparticles are each zinc oxide.

6. The composition of claim 1, wherein the metal particles comprise copper nanoparticles having an average particle size of from about 5 nm to about 500 nm.

7. The composition of claim 1, wherein a weight ratio of metal oxide microparticles to metal oxide nanoparticles is from 5:1 to 1:5.

8. The composition of claim 1, wherein a weight ratio of the sum of the metal oxide microparticles and metal oxide nanoparticles to the metal particles is from 8:1 to 99:1.

9. The composition of claim 1, wherein composition further comprises a polysiloxane.

10. The composition of claim 9, wherein the polysiloxane comprises a polydimethylsiloxane, a polydiethylsiloxane, a polydipropylsiloxane, or a polydiphenylsiloxane.

11. The composition of claim 1, wherein the silane compound is an alkyl trialkoxy silane.

12. The composition of claim 1, wherein the composition further comprises an organic solvent.

13. The composition of claim 1, wherein the composition does not include a perfluorolkylsiloxane.

14. A composition produced by mixing metal oxide nanoparticles, metal oxide microparticles, metal particles selected from the group consisting of silver, copper, strontium, selenium, zinc, iron, nickel, and any combination thereof, and a silane compound in a solvent.

15. An article comprising the composition of claim 1.

16. The article of claim 15, wherein the article comprises at least one surface comprising a coating of the composition or one or more components of the article are fabricated with the composition.

17. The article of claim 15, wherein the article comprises a medical device.

18. The article of claim 15, wherein the article is a catheter, a cannula, tubing, a suture, an artificial heart valve, a graft, a stent, circuitry, a joint prostheses, or a dressing.

19. A method for treating a wound, the method comprising applying the article of claim 15 to the wound.

20. A method for preventing the growth of bacteria on an article, the adhesion of fibrinogen on an article, or the adhesion of platelets on an article, the method comprising applying the coating composition of claim 15 to at least one surface of the article.
